(19) United States
(12) Patent Application Publication
(10) Pub. No.: US 2021/0182708 A1
(43) Pub. Date: Jun. 17, 2021

(54) TIME SERIES DATA PROCESSING DEVICE AND OPERATING METHOD THEREOF

(71) Applicant: ELECTRONICS AND TELECOMMUNICATIONS RESEARCH INSTITUTE, Daejeon (KR)

(72) Inventors: Hwin Dol PARK, Daejeon (KR); Jae Hun CHOI, Daejeon (KR); Youngwoong HAN, Daejeon (KR)

(21) Appl. No.: 17/116,767

(22) Filed: Dec. 9, 2020

(30) Foreign Application Priority Data
Dec. 11, 2019 (KR) 10-2019-0164359

(51) Int. Cl. G06N 5/04; G06N 20/00

(52) U.S. Cl. CPC G06N 5/04; G06N 20/00

(57) ABSTRACT

Disclosed are a time series data processing device and an operating method thereof. The time series data processing device includes a preprocessor, a learner, and a predictor. The preprocessor generates preprocessed data and interval data. The learner may adjust a feature weight, a time series weight, and a weight group of a feature distribution model for generating a prediction distribution, based on the interval data and the preprocessed data. The predictor may generate a feature weight, based on the interval data and the preprocessed data, may generate a time series weight, based on the feature weight and the interval data, and may calculate a prediction result and a reliability of the prediction result, based on the time series weight.

### CROSS-REFERENCE TO RELATED APPLICATIONS

This application claims priority under 35 U.S.C. § 119 to Korean Patent Application No. 10-2019-0164359 filed on Dec. 11, 2019, in the Korean Intellectual Property Office, the disclosures of which are incorporated by reference herein in their entireties.

### BACKGROUND

Embodiments of the present disclosure described herein relate to processing of time series data, and more particularly, relate to a time series data processing device that learns or uses a prediction model, and an operating method thereof.

The development of various technologies, including medical technology, improves the standard of human living and extends the human lifespan. However, changes in lifestyle and wrong eating habits according to technological development are causing various diseases. To lead a healthy life, there is a demand for predicting future health conditions beyond curing current diseases. Accordingly, a method of predicting health conditions in the future by analyzing a trend of time series medical data over time is proposed.

Advances in industrial technology and information and communication technologies allow information and data on a significant scale to be created. In recent years, technologies such as artificial intelligence for providing various services have emerged by learning electronic devices such as computers using such a large number of information and data. In particular, to predict future health conditions, a method of constructing a prediction model using various time series medical data is proposed. For example, time series medical data differs from data collected in other fields in that they have irregular time intervals, and complex and unspecified characteristics. Therefore, to predict future health conditions, there is a demand for effectively processing and analyzing the time series medical data.

### SUMMARY

Embodiments of the present disclosure provide a time series data processing device, which improves an accuracy of a prediction result that decreases depending on an irregular time of time series data, and an operating method thereof.

Embodiments of the present disclosure provide a time series data processing device, which provides an explainable prediction result by providing a basis and a validity for a prediction process of time series data, and an operating method thereof.

According to an embodiment of the present disclosure, a time series data processing device includes a preprocessor and a learner. The preprocessor generates interval data, based on a difference among each of a plurality of times on the basis of a last time of time series data, and generates preprocessed data of the time series data. The learner adjusts a feature weight depending on a time and a feature of the time series data, based on the interval data and the preprocessed data, a time series weight depending on a correlation between the plurality of times and the last time, and a weight group of a feature distribution model for generating a prediction distribution of the time series data corresponding to the last time. The weight group includes a first parameter for generating the feature weight, a second parameter for generating the time series weight, and a third parameter for generating the feature distribution model.

According to one embodiment, the preprocessor may generate the preprocessed data by adding an interpolation value to a missing value of the time series data, and may further generate masking data that distinguishes the missing value, and the learner may adjust the weight group, further based on the masking data.

According to one embodiment, the learner may include a feature learner that calculates the feature weight, based on the interval data, the preprocessed data, and the first parameter, and generates a first learning result, based on the feature weight, a time series learner that calculates the time series weight, based on the interval data, the first learning result, and the second parameter, and generates a second learning result, based on the time series weight, and a distribution learner that generates the prediction distribution, based on the second learning result and the third parameter, and the learner may adjust the weight group, based on the first learning result, the second learning result, and the prediction distribution.

According to one embodiment, the feature learner may include a missing value processor that generates first correction data of the preprocessed data, based on masking data that distinguishes a missing value of the preprocessed data, a time processor that generates second correction data of the preprocessed data, based on the interval data, a feature weight calculator that calculates the feature weight, based on the first parameter, the first correction data, and the second correction data, and a feature weight applier that generates the first learning result by applying the feature weight to the preprocessed data.

According to one embodiment, the time series learner may include a time series weight calculator that calculates the time series weight, based on the interval data, the first learning result, and the second parameter, and a time series weight applier that generates the second learning result by applying the time series weight to the preprocessed data.

According to one embodiment, the distribution learner may include a latent variable calculator that calculates a latent variable, based on the second learning result, and a multiple distribution generator that generates the prediction distribution, based on the latent variable.

According to one embodiment, the learner may encode a result obtained by applying the feature weight to the preprocessed data, and may calculate the time series weight, based on a correlation between the encoded result and the last time and a correlation between the encoded result and an encoded result of the last time.

According to one embodiment, the learner may calculate a coefficient of the prediction distribution, an average of the prediction distribution, and a standard deviation of the prediction distribution, based on a learning result obtained by applying the time series weight to the preprocessed data. According to one embodiment, the learner may calculate a conditional probability of a prediction result for the preprocessed data on the basis of the prediction distribution, based on the coefficient, the average, and the standard deviation, and may adjust the weight group, based on the conditional probability.

According to an embodiment of the present disclosure, the time series data processing device includes a preprocessor and a predictor. The preprocessor generates interval data, based on a difference among each of a plurality of times of time series data on the basis of a prediction time, and generates preprocessed data of the time series data. The predictor generates a feature weight depending on a time and a feature of the time series data, based on the interval data and the preprocessed data, generates a time series weight depending on a correlation between the plurality of times and a last time, based on the feature weight and the interval data, and calculates a prediction result corresponding to the prediction time and a reliability of the prediction result, based on the time series weight.

According to one embodiment, the preprocessor may generate the preprocessed data by adding an interpolation value to a missing value of the time series data, and may further generate masking data that distinguishes the missing value, and the predictor may generate the feature weight, further based on the masking data.

According to one embodiment, the predictor may include a feature predictor that calculates the feature weight, based on the interval data, the preprocessed data, and a feature parameter, and generates a first result, based on the feature weight, a time series predictor that calculates the time series weight, based on the interval data, the first result, and a time series parameter, and generates a second result, based on the time series weight, and a distribution predictor that selects at least some of prediction distributions, based on the second learning result and a distribution parameter, and calculates the prediction result and the reliability, based on the selected prediction distributions.

According to one embodiment, the feature predictor may include a missing value processor that generates first correction data of the preprocessed data, based on masking data that distinguishes a missing value of the preprocessed data, a time processor that generates second correction data of the preprocessed data, based on the interval data, a feature weight calculator that generates calculate the feature weight, based on the feature parameter, the first correction data, and the second correction data, and a feature weight applier that generates the first result by applying the feature weight to the preprocessed data.

According to one embodiment, the time series predictor may include a time series weight calculator that calculates the time series weight, based on the interval data, the first result, and the time series parameter, and a time series weight applier that generates the second result by applying the time series weight to the preprocessed data.

According to one embodiment, the distribution predictor may include a latent variable calculator that calculates a latent variable, based on the second result, a prediction value calculator that selects at least some of the prediction distributions, based on the latent variable, and calculates the prediction result, based on an average and a standard deviation of the selected prediction distributions, and a reliability calculator that calculates the reliability, based on the standard deviation of the selected prediction distributions.

According to one embodiment, the predictor may encode a result obtained by applying the feature weight to the preprocessed data, and may calculate the time series weight, based on a correlation between the encoded result and the prediction time and a correlation between the encoded result and an encoded result of the prediction time.

According to one embodiment, the predictor may calculate coefficients, averages, and standard deviations of prediction distributions, based on a result obtained by applying the time series weight to the preprocessed data, may select at least some of the prediction distributions by sampling the coefficients, and may generate the prediction result, based on the averages and the standard deviations of the selected prediction distributions.

According to an embodiment of the present disclosure, a method of operating a time series data processing device includes generating preprocessed data obtained by preprocessing time series data, generating interval data, based on a difference among each of a plurality of times of the time series data, on the basis of a prediction time, generating a feature weight depending on a time and a feature of the time series data, based on the preprocessed data and the interval data, generating a time series weight depending on a correlation between the plurality of times and the prediction time, based on a result of applying the feature weight and the interval data, and generating characteristic information of prediction distributions, based on a result of applying the time series weight.

According to one embodiment, the prediction time may be a last time of the time series data, and the method may further include calculating a conditional probability of a prediction result for the preprocessed data, based on the characteristic information, and adjusting a weight group of a feature distribution model for generating the prediction distributions, based on the conditional probability.

According to one embodiment, the method may further include calculating a prediction result corresponding to the prediction time, based on the characteristic information, and calculating a reliability of the prediction result, based on the characteristic information.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating a time series data processing device according to an embodiment of the present disclosure.

FIG. 2 is a diagram describing a time series irregularity of time series data described in FIG. 1.

FIGS. 3 and 4 are block diagrams of a preprocessor of FIG. 1.

FIG. 5 is a diagram describing interval data of FIGS. 3 and 4.

FIG. 6 is a block diagram of a learner of FIG. 1.

FIGS. 7 to 10 are diagrams specifically illustrating a feature learner of FIG. 6.

FIG. 11 is a diagram specifically illustrating a time series learner of FIG. 6.

FIG. 12 is a graph describing a correlation in the process of generating a time series weight of FIG. 11.

FIG. 13 is a diagram specifically illustrating a distribution learner of FIG. 6.

FIG. 14 is a block diagram of a predictor of FIG. 1.

FIG. 15 is a block diagram of a time series data processing device of FIG. 1.

### DETAILED DESCRIPTION

Hereinafter, embodiments of the present disclosure will be described clearly and in detail such that those skilled in the art may easily carry out the present disclosure.

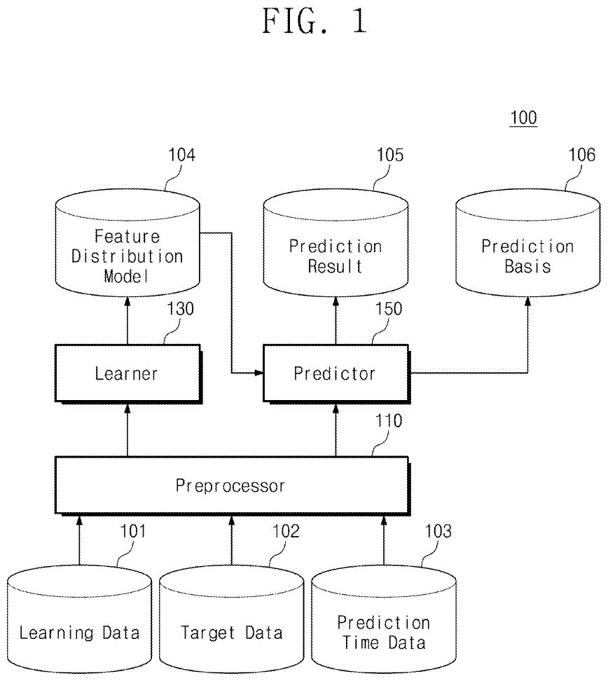

FIG. 1 is a block diagram illustrating a time series data processing device according to an embodiment of the present disclosure. A time series data processing device 100 of FIG. 1 will be understood as a configuration for preprocessing time series data and analyzing the preprocessed time series data to learn a prediction model, or to generate a prediction result. Referring to FIG. 1, the time series data processing device 100 includes a preprocessor 110, a learner 130, and a predictor 150.

The preprocessor 110, the learner 130, and the predictor 150 may be implemented in hardware, firmware, software, or a combination thereof. For example, software (or firmware) may be loaded into a memory (not illustrated) included in the time series data processing device 100 and may be executed by a processor (not illustrated). In an example, the preprocessor 110, the learner 130, and the predictor 150 may be implemented with hardware such as a dedicated logic circuit such as a Field Programmable Gate Array (FPGA) or an Application Specific Integrated Circuit (ASIC).

The preprocessor 110 may preprocess the time series data. The time series data may be a data set recorded over time and having a temporal order. The time series data may include at least one feature corresponding to each of a plurality of times arranged in time series. As an example, the time series data may include time series medical data representing health conditions of a user that are generated by diagnosis, treatment, or medication prescription in a medical institution, such as an electronic medical record (EMR). For clarity of explanation, the time series medical data are exemplarily described, but types of time series data are not limited thereto, and the time series data may be generated in various fields such as an entertainment, a retail, and a smart management.

The preprocessor 110 may preprocess the time series data to correct a time series irregularity, a missing value, and a type difference between features of the time series data. The time series irregularity means that time intervals among a plurality of times does not have regularity. The missing value is used to mean a feature that is missing or does not exist at a specific time among a plurality of features. The type difference between the features is used to mean that criteria for generating values are different for each feature. The preprocessor 110 may preprocess the time series data such that time series irregularities are reflected in the time series data, that missing values are interpolated, that the type between features is consistent. Details will be described later.

The learner 130 may learn a feature distribution model 104, based on the preprocessed time series data, that is, preprocessed data. The feature distribution model 104 may include a time series analysis model for calculating a prediction result in a future by analyzing the preprocessed time series data, and providing a prediction basis through distribution of prediction results. For example, the feature distribution model 104 may be constructed through an artificial neural network or a deep learning machine learning. To this end, the time series data processing device 100 may receive the time series data for learning from learning data 101. The learning data 101 may be implemented as a database in a server or storage medium outside or inside the time series data processing device 100. The learning data 101 may be implemented as the database, may be managed in a time series, and may be grouped and stored. The preprocessor 110 may preprocess the time series data received from the learning data 101 and may provide the preprocessed time series data to the learner 130. The preprocessor 110 may generate interval data by respectively calculating a difference between the times of the time series data, based on a last time of the learning data 101 to compensate for the time series irregularity of the learning data 101. The preprocessor 110 may provide the interval data to the learner 130.

The learner 130 may generate and adjust a weight group of the feature distribution model 104 by analyzing the preprocessed time series data. The learner 130 may generate a distribution of a prediction result through analysis of time series data, and may adjust the weight group of the feature distribution model 104 such that the generated distribution has a target conditional probability. The weight group may be a set of all parameters included a neural network structure or a neural network of a feature distribution model. The feature distribution model 104 may be implemented as a database in a server or a storage medium outside or inside the time series data processing device 100. The weight group and the feature distribution model may be implemented as the database, and may be managed and stored.

The predictor 150 may generate a prediction result by analyzing the preprocessed time series data. The prediction result may be a result corresponding to a prediction time such as a specific time in a future. To this end, the time series data processing device 100 may receive target data 102 and prediction time data 103 that are time series data for prediction. Each of the target data 102 and the prediction time data 103 may be implemented as a database in a server or a storage medium outside or inside the time series data processing device 100. The preprocessor 110 may preprocess the target data 102 and provide the preprocessed target data to the predictor 150. The preprocessor 110 may generate interval data by calculating a difference between the times of the time series data, based on the prediction time defined in the prediction time data 103 to compensate for the time series irregularity of the target data 102. The preprocessor 110 may provide the interval data to the predictor 150.

The predictor 150 may analyze the preprocessed time series data, based on the feature distribution model 104 learned from the learner 130. The predictor 150 may generate a prediction distribution by analyzing time series trends and features of the preprocessed time series data, and generate a prediction result 105 by sampling the prediction distribution. The predictor 150 may generate a prediction basis 106 by calculating a reliability of the prediction result 105, based on the prediction distribution. Each of the prediction result 105 and the prediction basis 106 may be implemented as a database in a server or a storage medium outside or inside the time series data processing device 100.

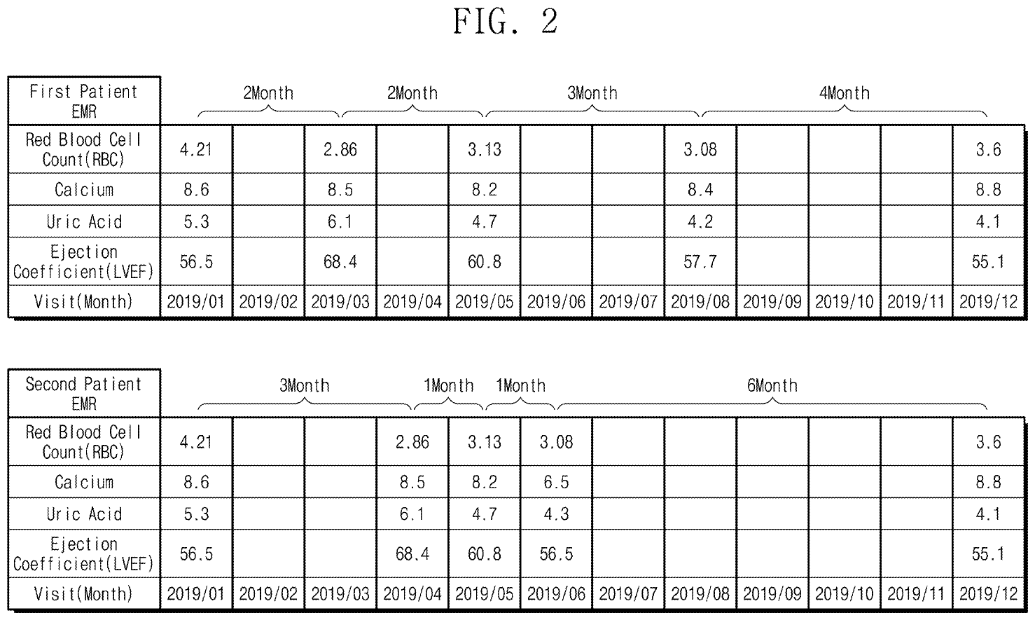

FIG. 2 is a diagram describing a time series irregularity of time series data described in FIG. 1. Referring to FIG. 2, medical time series data of a first patient and a second patient are illustrated. The time series data includes features such as red blood cell count, calcium, uric acid, and ejection coefficient.

Patient visits are irregular. Accordingly, the time series data may be generated, measured, or recorded at different visit times. Furthermore, when the prediction time of the time series data is not set, the time indicated by the prediction result is unclear. In general time series analysis, it is assumed that the time interval is uniform, such as data collected at a certain time through a sensor, and the prediction time is automatically set according to a regular time interval. This analysis may not consider irregular time intervals. The time series data processing device 100 of FIG. 1 may reflect the irregular time intervals and may provide a clear prediction time to perform learning and prediction. These specific details will be described later.

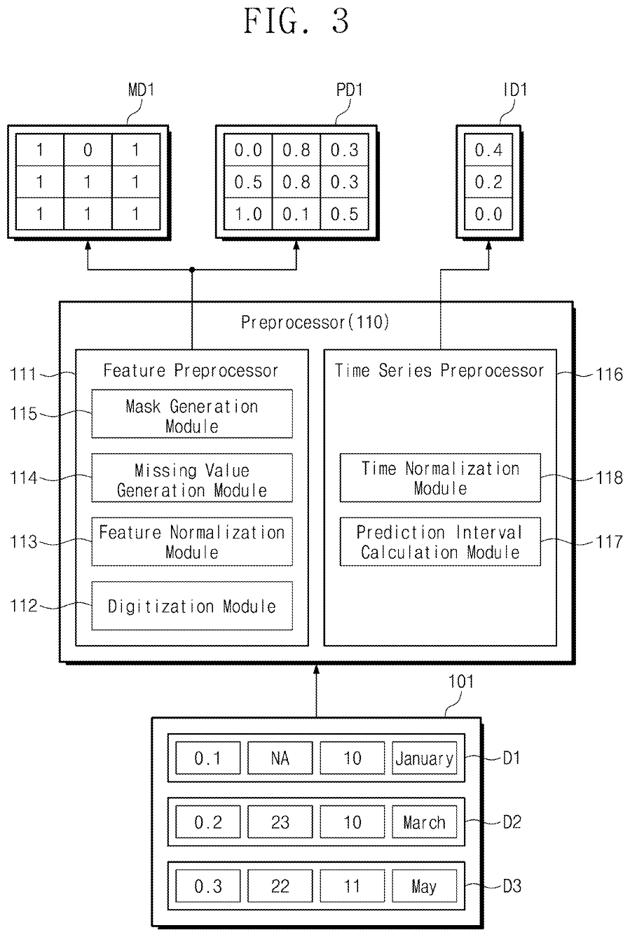

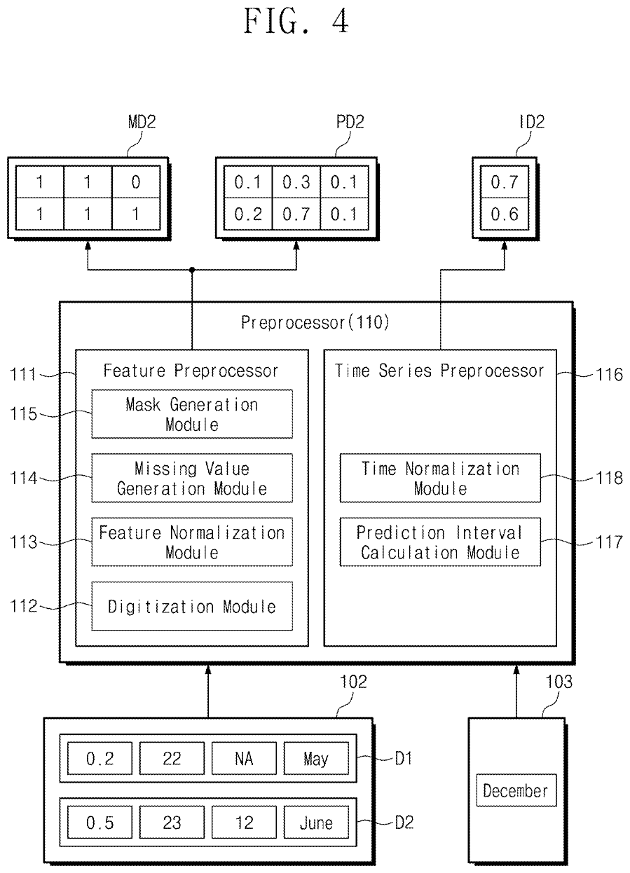

FIGS. 3 and 4 are block diagrams of a preprocessor of FIG. 1. FIG. 3 illustrates an operation in a learning operation of the preprocessor 110 of FIG. 1. FIG. 4 illustrates an operation in a prediction operation of the preprocessor 110 of FIG. 1.

Referring to FIG. 3, it will be understood as a configuration for preprocessing the learning data 101 which are time series data considering a presence of missing values and irregular time intervals. The preprocessor 110 may include a feature preprocessor 111 and a time series preprocessor 116. As described in FIG. 1, the feature preprocessor 111 and the time series preprocessor 116 may be implemented as hardware, firmware, software, or a combination thereof.

The feature preprocessor 111 and the time series preprocessor 116 receive the learning data 101. The learning data 101 may be data for learning the feature distribution model, or data for calculating the prediction result and the prediction basis through a learned feature distribution model. For example, the learning data 101 may include first to third data D1 to D3. Each of the first to third data D1 to D3 may include first to fourth features. In this case, the fourth feature may represent a time when each of the first to third data D1 to D3 is generated.

The feature preprocessor 111 may preprocess the learning data 101 to generate preprocessed data PD1. The preprocessed data PD1 may include features of the learning data 101 converted to have the same type. The preprocessed data PD1 may have features corresponding to first to third features of the learning data 101. The preprocessed data PD1 may be time series data obtained by interpolating a missing value NA. When the features of the learning data 101 have the same type and the missing value NA is interpolated, a time series analysis by the learner 130 or the predictor 150 of FIG. 1 may be easily performed. To generate the preprocessed data PD1, a digitization module 112, a feature normalization module 113, and a missing value generation module 114 may be implemented in the feature preprocessor 111.

The feature preprocessor 111 may generate masking data MD1 by preprocessing the learning data 101. The masking data MD1 may be data for distinguishing between the missing value NA and actual values of the learning data 101. The masking data MD1 may have values corresponding to first to third features for each of times of the learning data 101. The masking data MD1 may be generated so as not to treat the missing value NA as the same importance as the actual value during the time series analysis. To generate the masking data MD1, a mask generation module 115 may be implemented in the feature preprocessor 111.

The digitization module 112 may convert a type of non-numeric features in the learning data 101 into a numeric type. The non-numeric type may include a code type or a categorical type (e.g., −, +, ++, etc.). For example, the EMR data may have a data type promised according to a specific disease, prescription, or test, but may have a type in which the numeric type and the non-numeric type are mixed. The digitization module 112 may convert features of the non-numeric type of the learning data 101 into a numeric type. As an example, the digitization module 112 may digitize the features through an embedding method such as Word2Vec.

The feature normalization module 113 may convert values of the learning data 101 into values of a reference range. For example, the reference range may include values between 0 to 1, or −1 to 1. The learning data 101 may have a value in an independent range depending on the features. For example, a third feature of each of the first to third data D1 to D3 has numerical values 10, 10, and 11 outside the reference range. The feature normalization module 113 may normalize the third features 10, 10, and 11 of the learning data 101 to the same reference range as third features 0.3, 0.3, and 0.5 of the preprocessed data PD1.

The missing value generation module 114 may add an interpolation value to the missing value NA of the learning data 101. The interpolation value may have a preset value or may be generated based on another value of the learning data 101. For example, the interpolation value may have ‘0’, a median value or an average value of features at different times, or a feature value at adjacent times. For example, a second feature of the first data D1 has the missing value NA. The missing value generation module 114 may set the interpolation value as the second feature value of the second data D2 temporally adjacent to the first data D1.

The mask generation module 115 generates the masking data MD1, based on the missing value NA. The mask generation module 115 may generate the masking data MD1 by differently setting a value corresponding to the missing value NA and a value corresponding to other values (i.e., actual values). For example, the value corresponding to the missing value NA may be ‘0’, and the value corresponding to the actual value may be ‘1’.

The time series preprocessor 116 may preprocess the learning data 101 to generate interval data ID1. The interval data ID1 may include time interval information between the last time of the learning data 101 and times corresponding to the first to third data D1 to D3. In this case, the last time may mean a last time among the times indicated in the learning data 101. For example, May corresponding to the third data D3 may represent the last time. The interval data ID1 may have the same number of values as the learning data 101 in a time dimension. The interval data ID1 may be generated to consider the time series irregularity during the time series analysis. To generate the interval data ID1, a prediction interval calculation module 117 and a time normalization module 118 may be implemented in the time series preprocessor 116.

The prediction interval calculation module 117 may calculate the irregularity of the learning data 101. The prediction interval calculation module 117 may calculate a time interval, based on a difference between the last time and each of a plurality of times of the time series data. For example, based on May indicated by the third data D3, the first data D1 has a difference of 4 months, the second data D2 has a difference of 2 months, and the third data D3 has a difference of 0 month. The prediction interval calculation module 117 may calculate this time difference.

The time normalization module 118 may normalize an irregular time difference calculated from the prediction interval calculation module 117. The time normalization module 118 may convert a value calculated from the prediction interval calculation module 117 into a value in a reference range. For example, the reference range may include a value between 0 to 1, or −1 to 1. Times quantified by year, month, day, etc. may deviate from the reference range, and the time normalization module 118 may normalize the time to the reference range. As a result of normalization, values of the interval data ID1 corresponding to each of the first to third data D1 to D3 may be generated.

Referring to FIG. 4, it will be understood as a configuration for preprocessing the target data 102 that is time series data in consideration of a presence of missing values and irregular time intervals. The preprocessor 110 may include the feature preprocessor 111 and the time series preprocessor 116. As described in FIG. 1, the feature preprocessor 111 and the time series preprocessor 116 may be implemented as hardware, firmware, software, or a combination thereof.

To generate preprocessed data PD2 and masking data MD2, the digitization module 112, the feature normalization module 113, the missing value generation module 114, and the mask generation module 115 may be implemented in the feature preprocessor 111. A process of generating the preprocessed data PD2 and the masking data MD2 is substantially the same as the process of generating the preprocessed data PD1 and the masking data MD1 by the feature preprocessor 111 of FIG. 3.

The time series preprocessor 116 may preprocess the target data 102 to generate interval data ID2. The interval data ID2 may include time interval information between the prediction time and times corresponding to the first and second data D1 and D2. In this case, the prediction time may be defined by the prediction time data 103. For example, December may represent the prediction time according to the prediction time data 103. Thus, under time series irregularities, a clear prediction time may be provided. To generate the interval data ID2, the prediction interval calculation module 117 and the time normalization module 118 may be implemented in the time series preprocessor 116.

The prediction interval calculation module 117 may calculate a time interval, based on a difference between the prediction time and each of a plurality of times of the time series data. For example, as of December, the first data D1 has a difference of 7 months, and the second data D2 has a difference of 6 months. The prediction interval calculation module 117 may calculate this time difference. The time normalization module 118 may normalize the irregular time difference calculated from the prediction interval calculation module 117. As a result of normalization, values of the interval data ID2 corresponding to each of the first and second data D1 and D2 may be generated.

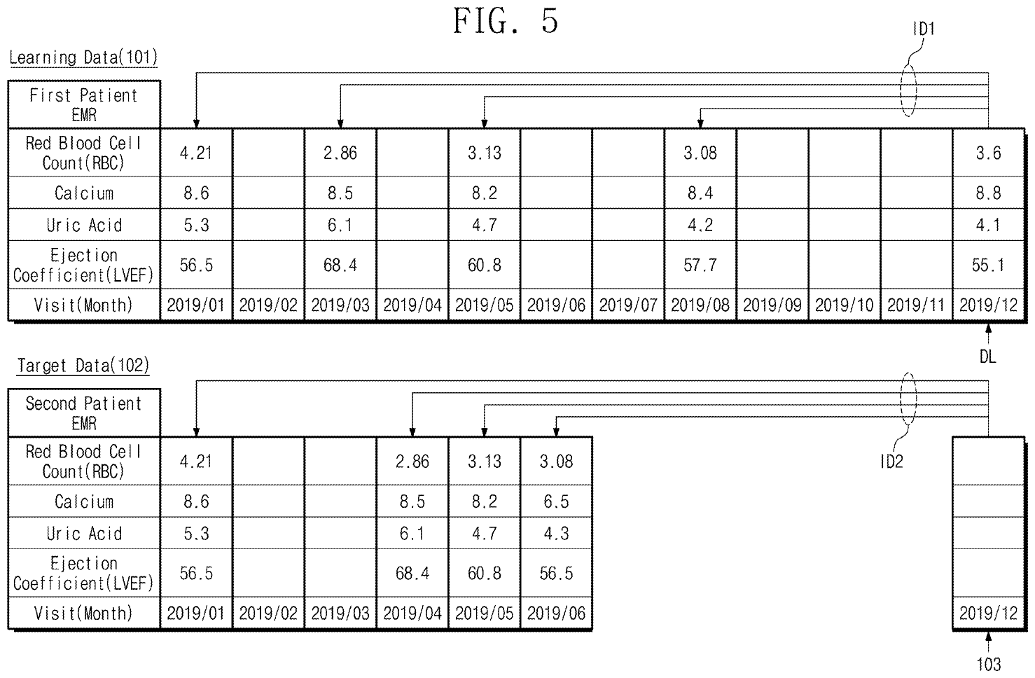

FIG. 5 is a diagram describing interval data of FIGS. 3 and 4. Referring to FIG. 5, a criterion for generating the interval data ID1 from the learning data 101 and a criterion for generating the interval data ID2 from the target data 102 are different from each other. For example, the learning data 101 and the target data 102 are described as the medical time series data of a first patient and a second patient. The time series data includes features such as red blood cell count, calcium, uric acid, and ejection coefficient.

The criterion for generating the interval data ID1 from the learning data 101 is the last time of the time series data. That is, based on the time series data of the first patient, December 2019, which is the time corresponding to the last data DL, is the last time. Based on the last time, a time interval of times at which features are generated may be calculated. As a result of the calculation, the interval data ID1 are generated.

The criterion for generating the interval data ID2 from the target data 102 is a prediction time. That is, December 2019 set in the prediction time data 103 is the prediction time. Based on the prediction time, the time interval of times at which features are generated may be calculated. As a result of the calculation, the interval data ID2 are generated.

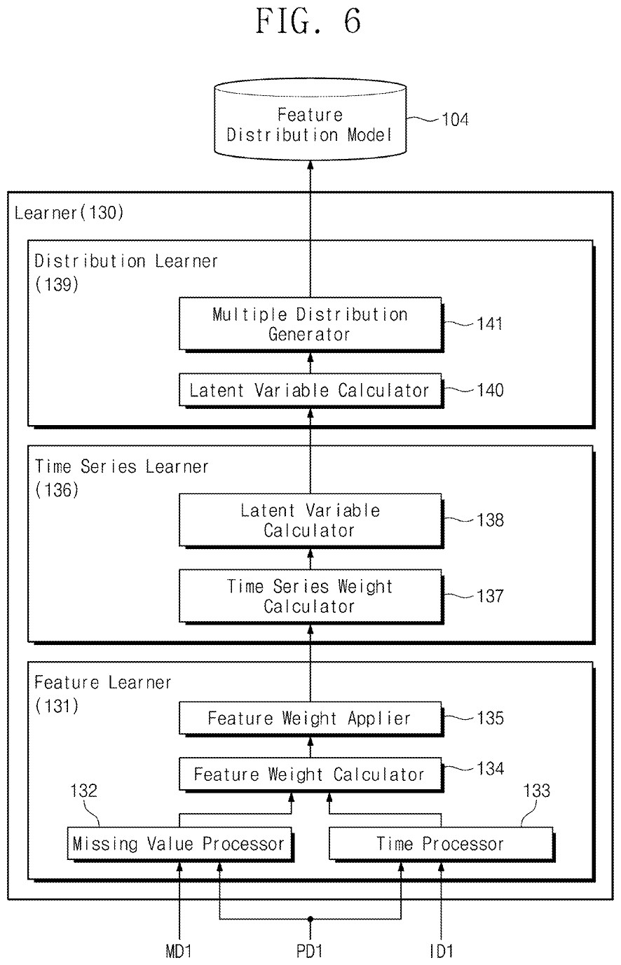

FIG. 6 is a block diagram of a learner of FIG. 1. The block diagram of FIG. 6 will be understood as a configuration for learning the feature distribution model 104 and determining a weight group, based on the preprocessed data PD1. Referring to FIG. 6, the learner 130 may include a feature learner 131, a time series learner 136, and a distribution learner 139. As described in FIG. 1, the feature learner 131, the time series learner 136, and the distribution learner 139 may be implemented as hardware, firmware, software, or a combination thereof.

The feature learner 131 analyzes a time and a feature of the time series data, based on the preprocessed data PD1, the masking data MD, and the interval data ID that are generated from the preprocessor 110 of FIG. 3. The feature learner 131 may generate parameters for generating a feature weight by learning at least a part of the feature distribution model 104. These parameters (feature parameters) are included in the weight group. The feature weight depends on the time and feature of the time series data.

The feature weight may include a weight of each of a plurality of features corresponding to a specific time. That is, the feature weight may be understood as an index that determines the importance of values included in the time series data that are calculated based on the feature parameter. To this end, a missing value processor 132, a time processor 133, a feature weight calculator 134, and a feature weight applier 135 may be implemented in the feature learner 131.

The missing value processor 132 may generate first correction data for correcting an interpolation value of the preprocessed data PD1, based on the masking data MD1. Alternatively, the missing value processor 132 may generate the first correction data by applying the masking data MD1 to the preprocessed data PD1. As described above, the interpolation value may be a value obtained by replacing the missing value with another value. The learner 130 may not know whether the values included in the preprocessed data PD1 are randomly assigned interpolation values or actual values. Accordingly, the missing value processor 132 may generate the first correction data for adjusting the importance of the interpolation value by using the masking data MD.

The time processor 133 may generate second correction data for correcting the irregularity of the time interval of the preprocessed data PD1, based on the interval data ID1. Alternatively, the time processor 133 may generate the second correction data by applying the interval data ID1 to the preprocessed data PD1. The time processor 133 may generate the second correction data for adjusting the importance of each of a plurality of times corresponding to the preprocessed data PD1 by using the interval data ID1. That is, the features corresponding to a specific time may be corrected with the same importance by the second correction data.

The feature weight calculator 134 may calculate the feature weight corresponding to features and times of the preprocessed data PD1, based on the first correction data and the second correction data. The feature weight calculator 134 may apply the importance of the interpolation value and the importance of each of the times to the feature weight. For example, the feature weight calculator 134 may use an attention mechanism to generate the feature weight such that the prediction result pays attention to the specified feature.

The feature weight applier 135 may apply the feature weight calculated from the feature weight calculator 134 to the preprocessed data PD1. As a result of application, the feature weight applier 135 may generate a first learning result in which the complexity of time and feature is applied to the preprocessed data PD1. For example, the feature weight applier 135 may multiply the feature weight corresponding to a specific time and a feature by a corresponding feature of the preprocessed data PD1. However, the present disclosure is not limited thereto, and the feature weight may be applied to an intermediate result of analyzing the preprocessed data PD1 by the first or second correction data.

The time series learner 136 analyzes a correlation between the plurality of times and the last time and a correlation between the plurality of times and the first learning result of the last time, based on the first learning result generated from the feature weight applier 135. When the feature learner 131 analyzes values corresponding to the feature and the time (in this case, the time may mean a specific time in which time intervals are reflected) of the time series data, the time series learner 136 may analyze a trend of data over time or a correlation between the prediction time and the specific time. The time series learner 136 may generate parameters for generating the time series weight by learning at least a part of the feature distribution model 104. These parameters (i.e., time series parameters) are included in the weight group.

The time series weight may include a weight of each of a plurality of times of time series data. That is, the time series weight may be understood as an index that determines the importance of each time of the time series data, which is calculated based on the time series parameter. To this end, a time series weight calculator 137 and a time series weight applier 138 may be implemented in the time series learner 136.

The time series weight calculator 137 may calculate a time series weight corresponding to times of the first learning result generated by the feature learner 131. The time series weight calculator 137 may apply the importance of each of the times to the time series weight, based on the last time. The time series weight calculator 137 may apply the importance of each of the times to the time series weight, based on the learning result of the last time. For example, the time series weight calculator 137 may generate the time series weight by scoring a correlation between a plurality of times and the last time and a correlation between the plurality of times and the first learning result of the last time.

The time series weight applier 138 may apply the time series weight calculated from the time series weight calculator 137 to the preprocessed data PD1. As a result of the application, the time series weight applier 138 may generate a second learning result in which an irregularity of the time interval and a time series trend are applied. For example, the time series weight applier 138 may multiply the time series weight corresponding to a specific time by features of the first learning result corresponding to the specific time. However, the present disclosure is not limited thereto, and the time series weight may be applied to the first learning result or the intermediate result that is obtained by analyzing the first learning result.

The distribution learner 139 analyzes a conditional probability of prediction distributions for calculating the prediction result and the reliability of the prediction result, based on the second learning result generated from the time series weight applier 138. The distribution learner 139 may generate various distributions to describe the prediction basis of the prediction result. The distribution learner 139 may analyze the conditional probability of the prediction result of the learning data, based on the prediction distributions. The distribution learner 139 may generate parameters for generating prediction distributions by learning at least a part of the feature distribution model 104. These parameters (i.e., distribution parameters) are included in the weight group. To this end, a latent variable calculator 140 and a multiple distribution generator 141 may be implemented in the distribution learner 139.

The latent variable calculator 140 may generate a latent variable for the second learning result generated from the time series learner 136. In this case, the latent variable will be understood as the intermediate result that is obtained by analyzing the second learning result to easily generate various prediction distributions, and may be expressed as feature vectors.

The multiple distribution generator 141 may generate the prediction distributions by using the latent variable calculated from the latent variable calculator 140. The multiple distribution generator 141 may generate characteristic information such as coefficients, averages, and standard deviations of each of the prediction distributions by using the latent variable. The multiple distribution generator 141 may calculate the conditional probability of the prediction result for the preprocessed data PD1 or the learning data, based on the prediction distributions, using the generated coefficients, averages, and standard deviations. Based on the calculated conditional probability, the weight group may be adjusted, and the feature distribution model 104 may be learned. Using the feature distribution model 104, a prediction result for target data is calculated in a later prediction operation, and a prediction basis including a reliability of the prediction result may be provided.

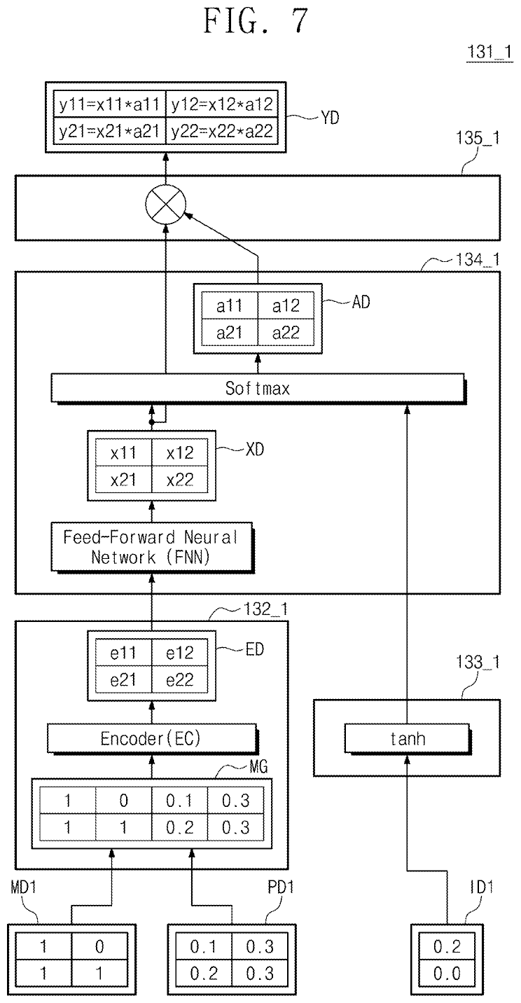

FIGS. 7 to 10 are diagrams specifically illustrating a feature learner of FIG. 6. Referring to FIGS. 7 to 10, the feature learners 131_1 to 131_4 may be implemented with missing value processors 132_1 to 132_4, time processors 133_1 to 133_4, feature weight calculators 134_1 to 134_4, and feature weight appliers 135_1 to 135_4.

Referring to FIG. 7, the missing value processor 132_1 may generate merged data MG by merging the masking data MD1 and the preprocessed data PD1. The missing value processor 132_1 may generate encoded data ED by encoding the merged data MG. For encoding, the missing value processor 132_1 may include an encoder EC. For example, the encoder EC may be implemented as a 1D convolution layer or an auto-encoder. A weight and a bias for this encoding may be included in the above-described feature parameter, and may be generated by the learner 130. The encoded data ED correspond to the first correction data described in FIG. 6.

The time processor 133_1 may model the interval data ID1. For example, the time processor 133_1 may model the interval data ID1 by using a nonlinear function such as ‘tanh’. In this case, the weight and the bias may be applied to the corresponding function. For example, the time processor 133_1 may model the interval data ID1 through the ‘tank’ function. The weight and bias may be included in the above-described feature parameter, and may be generated by the learner 130. The modeled interval data ID1 correspond to the second correction data described in FIG. 6.

The feature weight calculator 134_1 may generate a feature weight AD such that a prediction result focuses on a specified feature using the attention mechanism. In addition, the feature weight calculator 134_1 may process the modeled interval data together such that the feature weight AD reflects the time interval of the time series data. For example, the feature weight calculator 134_1 may analyze features of the encoded data ED through a feed-forward neural network. The encoded data ED may be correction data in which the importance of the missing value is reflected in the preprocessed data PD1 by the masking data MD1. The feed-forward neural network may analyze the encoded data ED, based on the weight and the bias. This weight and the bias may be included in the above-described feature parameters and may be generated by the learner 130. The feature weight calculator 134_1 may generate feature analysis data XD by analyzing the encoded data ED.

The feature weight calculator 134_1 may calculate the feature weight AD by applying the feature analysis data XD and the modeled interval data to the ‘softmax’ function. In this case, the weight and the bias may be applied to the corresponding function. The weight and bias may be included in the above-described feature parameter, and may be generated by the learner 130.

The feature weight applier 135_1 may apply the feature weight AD to the feature analysis data XD. For example, the feature weight applier 135_1 may generate a first learning result YD by multiplying the feature weight AD by the feature analysis data XD. However, the present disclosure is not limited thereto, and the feature weight AD may be applied to the preprocessed data PD1 instead of the feature analysis data XD.

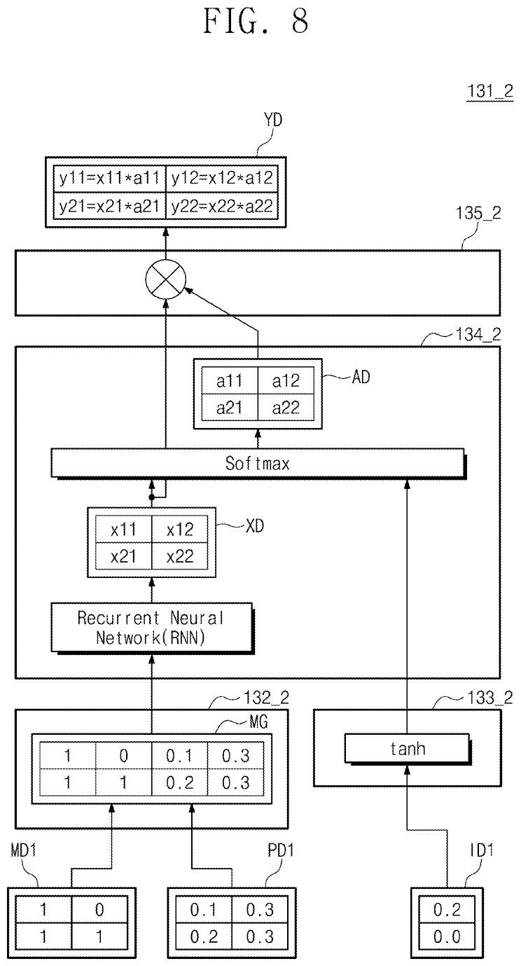

Referring to FIG. 8, the feature learner 131_2 may operate substantially the same as the feature learner 131_1 of FIG. 7 except for the missing value processor 132_2 and the feature weight calculator 134_2. Configurations that operate substantially the same are omitted from the description.

The missing value processor 132_2 may generate merged data MG by merging the masking data MD1 and the preprocessed data PD1. Unlike FIG. 7, the missing value processor 132_2 may not postprocess the merged data MG. For example, the feature weight calculator 134_2 may analyze the merged data MG through a recurrent neural network instead of the feed-forward neural network. The recurrent neural network may additionally perform a function of encoding the merged data MG. The recurrent neural network may analyze the merged data MG, based on the weight and bias.

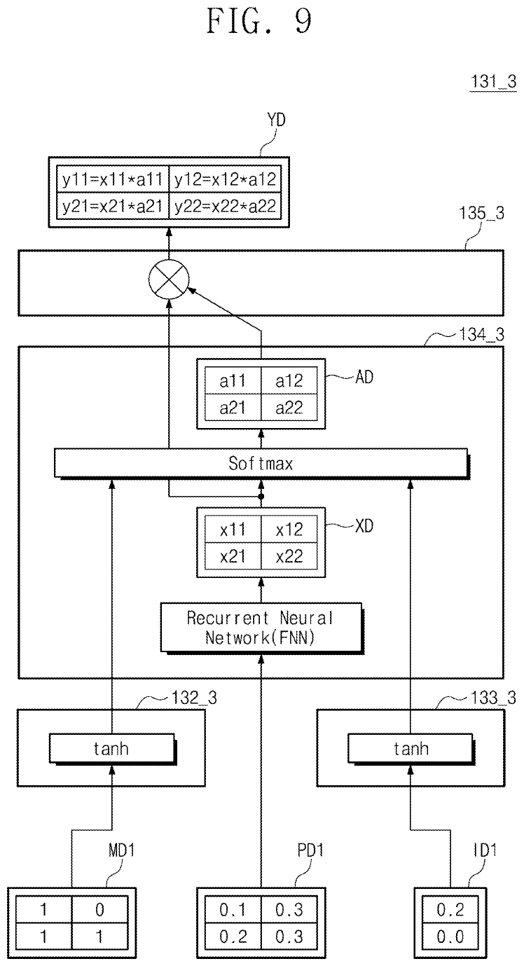

Referring to FIG. 9, the feature learner 131_3 may operate substantially the same as the feature learner 131_1 of FIG. 7 except for the missing value processor 132_3 and the feature weight calculator 134_3. Configurations that operate substantially the same are omitted from the description.

The missing value processor 132_3 may model the masking data MD1. For example, the missing value processor 132_3 may model the masking data MD1 by using the nonlinear function such as ‘tanh’. In this case, the weight and the bias may be applied to the corresponding function. The weight and the bias may be included in the above-described feature parameter, and may be generated by the learner 130.

The feature weight calculator 134_3 may process the modeled masking data, similar to the modeled interval data, using the attention mechanism. The feature weight calculator 134_3 may analyze features of the preprocessed data PD1 and generate the feature analysis data XD through the feed-forward neural network. The feature weight calculator 134_3 may calculate the feature weight AD by applying the feature analysis data XD, the modeled masking data, and modeled interval data to the ‘softmax’ function.

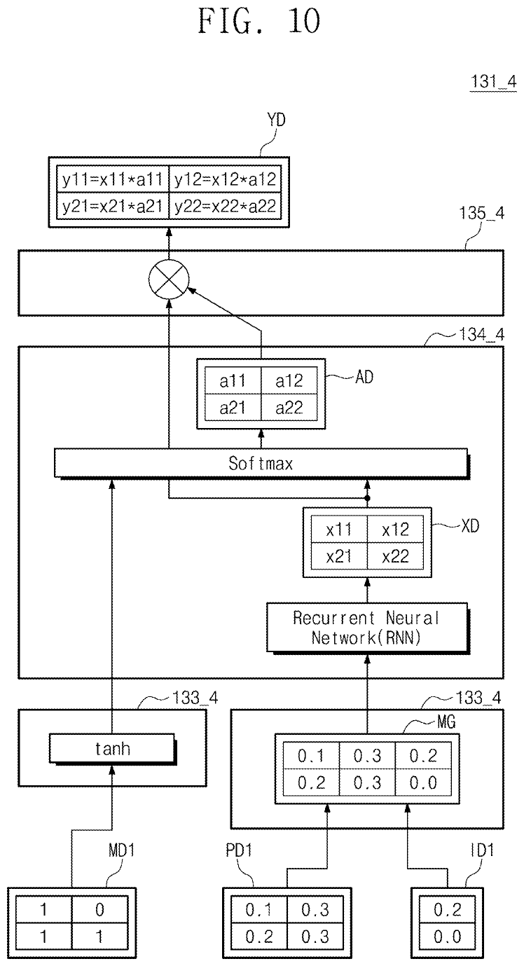

Referring to FIG. 10, the feature learner 131_4 may operate substantially the same as the feature learner 131_1 of FIG. 7 except for the time processor 133_4 and the feature weight calculator 134_4. Configurations that operate substantially the same are omitted from the description.

The time processor 133_4 may generate the merged data MG by merging the interval data ID1 and the preprocessed data PD1. The feature weight calculator 134_4 may analyze the merged data MG through the feed-forward neural network. The recurrent neural network may analyze merged data MG and generate the feature analysis data XD, based on the weight and the bias. The feature weight calculator 134_4 may calculate the feature weight AD by applying the feature analysis data XD and the modeled masking data to the ‘softmax’ function.

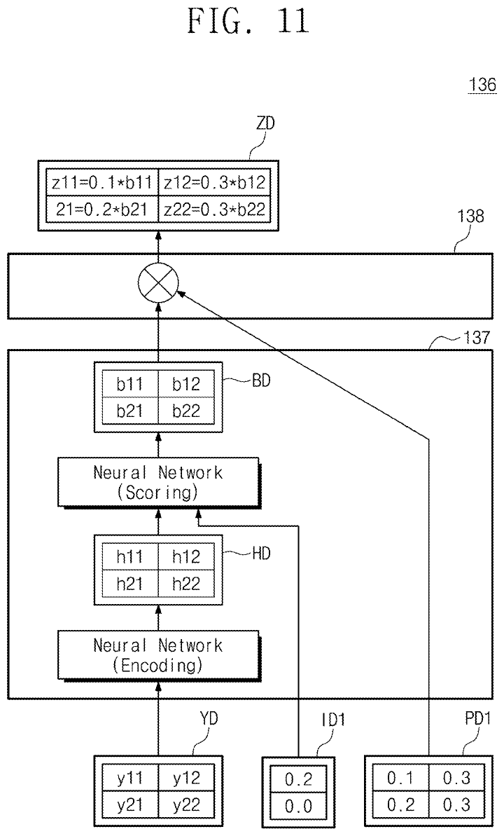

FIG. 11 is a diagram specifically illustrating a time series learner of FIG. 6. Referring to FIG. 11, the time series learner 136 may be implemented with the time series weight calculator 137 and the time series weight applier 138.

The time series weight calculator 137 may generate encoded data HD by encoding the first learning result YD generated from the feature learner 131 described in FIGS. 6 to 10. For encoding, the time series weight calculator 137 may include an encoder. For example, the encoder may be implemented as a 1D convolution layer or an auto-encoder. The weight and bias for this encoding may be included in the above-described time series parameter and may be generated by the learner 130.

The time series weight calculator 137 may generate a time series weight BD based on the encoded data HD and the interval data ID1. The time series weight calculator 137 may calculate a first score by analyzing a correlation between the encoded data HD and a value of the encoded data HD corresponding to the last time. The time series weight calculator 137 may calculate a second score by analyzing a correlation between times of the encoded data HD and the last time. The time series weight calculator 137 may normalize the first and second scores and generate the time series weight by reflecting the weight. The time series weight calculator 137 may analyze a correlation between the encoded data HD and the last time or the last time value through a neural network (e.g., the feed-forward neural network). This process may be the same as in Equation 1.

$$score 1 = hiW ( {hL}^{T} ) \\ score 2 = hi ( W Δ {t}^{T} ) \\ {a}_{1} = align 1 ( hi , hL ) = sin ( norm ( score 1 ) ) , 0 < score 1 < \frac{π}{2} \\ {a}_{2} = align 2 ( hi , Δ t ) = cos ( norm ( score 2 ) ) , 0 < score 2 < \frac{π}{2} \\ bi = softmax ( W \sum {W}_{weithed suma} ) \qquad [ Equation 1 ]$$

Referring to Equation 1, the first score may be calculated based on a correlation between values ‘hi’ of encoded data and a value ‘hL’ of encoded data corresponding to the last time. The second score may be calculated based on a correlation between the values ‘hi’ of the encoded data and the last time. The first score is normalized between ‘0’ and ‘π/2’, and the ‘sin’ function may be applied such that as a score value increases, the weight increases. As a result of the application, a first value ‘a1’ may be generated. The second score is normalized between ‘0’ and ‘π/2’, and the ‘cos’ function may be applied such that as a score value increases, the weight decreases. As a result of the application, a second value ‘a2’ may be generated. The first value ‘a1’ and the second value a2′ are weighted and added, and may be applied to the ‘softmax’ function. As a result, a time series weight ‘bi’ may be generated. The weight ‘W’ for this may be included in the time series parameter and may be generated by the learner 130.

The time series weight applier 138 may apply the time series weight BD to the preprocessed data PD1. For example, the time series weight applier 138 may generate a second learning result ZD by multiplying the time series weight BD by the preprocessed data PD1. However, the present disclosure is not limited thereto, and the time series weight BD may be applied to the encoded data HD or the first learning result TD instead of the preprocessed data PD1.

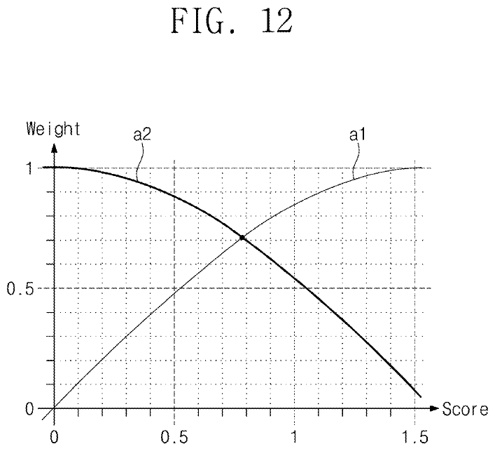

FIG. 12 is a graph describing a correlation in the process of generating a time series weight of FIG. 11. Referring to FIG. 12, a horizontal axis may be defined as the score (first score, second score) described in FIG. 11, and a vertical axis may be defined as a median value (first value, second value) for generating the time series weight BD described in FIG. 11.

A correlation between values of encoded data of FIG. 11 corresponding to respective features of the time series data and a value of encoded data of the last time may be represented by the first score. The first score of values having a high correlation with the value of the last time may appear relatively higher. The first value ‘a1’ may be generated by applying the ‘sin’ function to the normalized first score. As a result, as the first score increases, the first value ‘a1’ may increase. Accordingly, values having a high correlation with the last time value may have a high importance in generating the time series weight BD.

A correlation between the values of the encoded data of FIG. 11 corresponding to each feature of the time series data and the last time may be represented by the second score. The second score of values corresponding to a time far from the last time may appear relatively higher. The second value ‘a2’ may be generated by applying the ‘cos’ function to the normalized second score. As a result, as the second score increases, the second value ‘a2’ may decrease. Accordingly, old values from the last time may have a low importance in generating the time series weight (BD).

As the time series weight BD is generated using the first value ‘a1’ and the second value ‘a2’, the time series weight BD may have a value depending on the correlation between a plurality of times of the time series data and the last time (prediction time). That is, the time series weight BD for each of the features may be generated in consideration of a temporal distance of the time series data on the basis of the last time and a relevance with data corresponding to the last time.

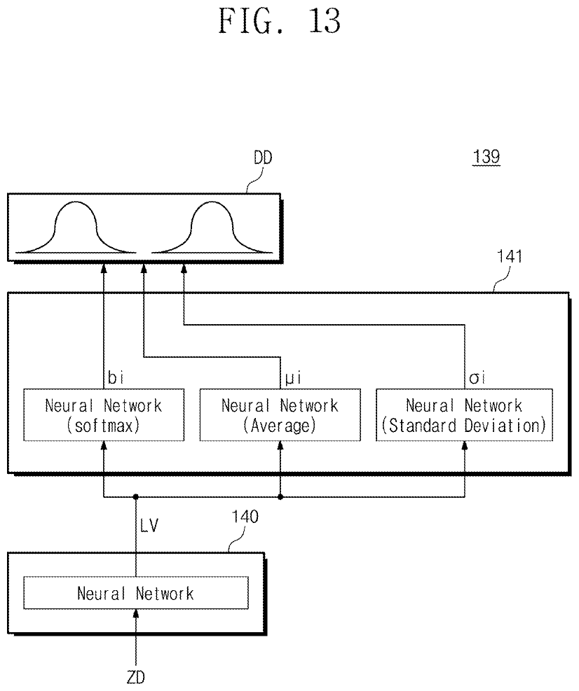

FIG. 13 is a diagram specifically illustrating a distribution learner of FIG. 6. Referring to FIG. 13, the distribution learner 139 may be implemented with the latent variable calculator 140 and the multiple distribution generator 141.

The latent variable calculator 140 may generate a latent variable LV for the second learning result generated from the time series learner 136. The latent variable calculator 140 may analyze the second learning result ZD through the neural network to easily generate various prediction distributions. The latent variable LV generated as a result of the analysis may be input to the multiple distribution generator 141. The weight and the bias for analysis of the neural network may be included in the above-described distribution parameter, and may be generated by the learner 130.

The multiple distribution generator 141 may transfer the latent variable LV to three neural networks. The multiple distribution generator 141 may generate a plurality of (e.g., ‘i’ pieces) prediction distributions DD for calculating the conditional probability of the prediction result for the learning data. To generate the prediction distributions DD, the latent variable LV may be input to the neural network for generating a coefficient ‘bi’ (mixing coefficient) of the prediction distributions DD. The neural network may generate the coefficient ‘bi’ by applying the latent variable LV to the ‘softmax’ function. Also, the latent variable LV may be input to a neural network for generating an average ‘μi’ of the prediction distributions DD. In addition, the latent variable LV may be input to a neural network for generating a standard deviation ‘σi’ of the prediction distributions DD. An exponential function may be used such that a negative number does not appear in a process of generating the standard deviation ‘σi’. The weight and the bias for generating the coefficient ‘bi’, the average ‘μi’, and the standard deviation ‘σi’ of neural networks may be included in the distribution parameter described above, and may be generated by the learner 130.

The distribution learner 139 may calculate the conditional probability of the prediction result of the preprocessed data PD1 or the learning data 101, based on the coefficient ‘bi’, the average ‘μi’, and the standard deviation ‘σi’ of the generated prediction distributions DD. This conditional probability may be calculated as in Equation 2.

$$p ( y \vert ϰ ) = \sum {b}_{i} ( ϰ ) N ( y \vert μ , σ ) ( ϰ ) \\ N ( μ , σ ) ( ϰ ) = \frac{1}{σ \sqrt{2 π}} exp ( - \frac{{( ϰ - μ )}^{2}}{2 {σ}^{2}} ) \qquad [ Equation 2 ]$$

Referring to Equation 2, ‘x’ is defined as a condition to be analyzed, such as the learning data 101 or preprocessed data PD1, and ‘y’ is defined as the corresponding prediction result. In the learning operation, the prediction result may be a value of the learning data 101 or preprocessed data PD1 corresponding to the last time. In the prediction operation, the prediction result may be a result of a prediction time defined by the set prediction time data 103. Equation 2 is an equation developed by assuming that the prediction distributions DD are Gaussian distributions, but the distributions of the prediction distributions DD are not limited to this normal distribution. As the coefficient ‘bi’, the average ‘μi’, and the standard deviation ‘σi’ of the prediction distributions DD are applied to Equation 2, the conditional probability p(y|x) may be calculated. Based on the calculated conditional probability p(y|x), the weight group may be adjusted, and the feature distribution model 104 may be learned.

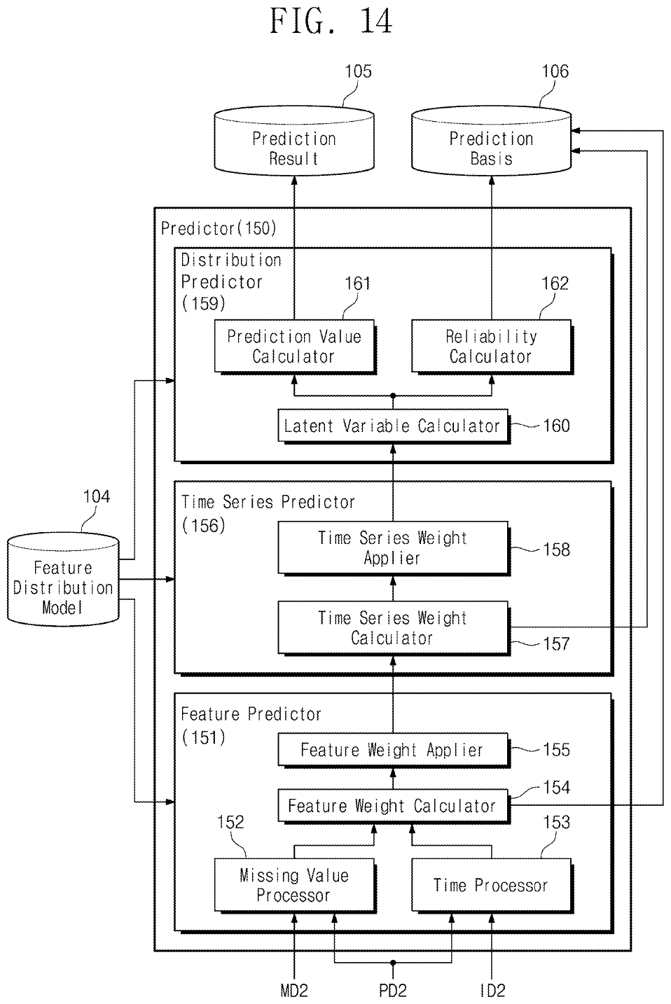

FIG. 14 is a block diagram of a predictor of FIG. 1. The block diagram of FIG. 14 will be understood as a configuration for analyzing the preprocessed data PD2 and generating the prediction result 105 and the prediction basis 106, based on the feature distribution model 104 and the weight group learned by the learner 130. Referring to FIG. 14, the predictor 150 may include a feature predictor 151, a time series predictor 156, and a distribution predictor 159. The feature predictor 151, the time series predictor 156, and the distribution predictor 159 may be implemented in hardware, firmware, software, or a combination thereof, as described in FIG. 1.

The feature predictor 151 analyzes the time and the feature of the time series data, based on the preprocessed data PD2, the masking data MD2, and the interval data ID2 generated from the preprocessor 110 of FIG. 4. In this case, the interval data ID2 are generated based on a difference between times of time series data on the basis of the prediction time data 103. A missing value processor 152, a time processor 153, a feature weight calculator 154, and a feature weight applier 155 may be implemented in the feature predictor 151, and may be implemented substantially the same as the missing value processor 132, the time processor 133, the feature weight calculator 134, and the feature weight applier 135 of FIG. 6. The feature predictor 151 may analyze the preprocessed data PD1, based on the feature parameter of the feature distribution model 104 and generate a first result.

The time series predictor 156 analyzes a correlation between a plurality of times and the last time and a correlation between the plurality of times and a first learning result of the last time, based on the first result generated from the feature predictor 151. A time series weight calculator 157 and a time series weight applier 158 may be implemented in the time series predictor 156, and may be implemented substantially the same as the time series weight calculator 137 and the time series weight applier 138 of FIG. 6. The time series predictor 156 may analyze the first result and generate a second result, based on the time series parameter provided from the feature distribution model 104.

The distribution predictor 159 may calculate the prediction result 105 corresponding to the prediction time, based on the second result generated from the time series predictor 156, and may further calculate the prediction basis 106 such as a reliability of the prediction result. A latent variable calculator 160, a prediction value calculator 161, and a reliability calculator 162 may be implemented in the distribution predictor 159. The latent variable calculator 160 may be implemented substantially the same as the latent variable calculator 140 of FIG. 6.

The prediction value calculator 161 may calculate characteristic information such as the coefficient, the average, and the standard deviation corresponding to prediction distributions, based on the latent variable. The prediction value calculator 161 may generate the prediction result 105 by using a sampling method based on the coefficient, the average, and the standard deviation. The prediction value calculator 161 may select some prediction distributions among various prediction distributions depending on the coefficient, the average, and the standard deviation, and may calculate the prediction result 105 by calculating an average of the selected distributions and an average of the standard deviations. The prediction result 105 may be calculated as in Equation 3.

$$index = Gumbel softmax sampling ( bi ) \\ {u}_{selected} = μ {i}_{( index )} \\ {σ}_{selected} = σ i ( index ) \\ Result = {\sum }_{n}^{ } \frac{( {u}_{selected} + {σ}_{selected} )}{n} \qquad [ Equation 3 ]$$

Referring to Equation 3, the prediction value calculator 161 may generate an index by sampling (e.g., Gumbel softmax sampling) the coefficient ‘bi’. Based on this index, some distributions of the various prediction distributions may be selected. Accordingly, as the average pi′ corresponding to the selected prediction distributions and the average of the standard deviation ‘σi’ (where, ‘n’ is the number of sampling) are calculated, the prediction result 105 may be calculated.

The reliability calculator 162 may calculate the standard deviation of selected prediction distributions when the prediction result 105 is calculated. Through this standard deviation, a standard error corresponding to the reliability of the prediction result 105 may be calculated. The reliability (standard error, SE), that is, the prediction basis 106 may be calculated as in Equation 4.

$$σ = {\sum }_{n}^{ } \frac{{σ}_{selected}}{n} \\ SE = \frac{σ}{\sqrt{n}} \qquad [ Equation 4 ]$$

Through Equation 4, the standard error SE of the prediction result 105 is calculated, and this standard error SE may be included in the prediction basis 106. Furthermore, the prediction basis 106 may further include a feature weight generated from the feature weight calculator 154 and a time series weight generated from the time series weight calculator 157. This may be to provide a basis and validity for a prediction process, and to provide the explainable prediction result 105 to a user, etc.

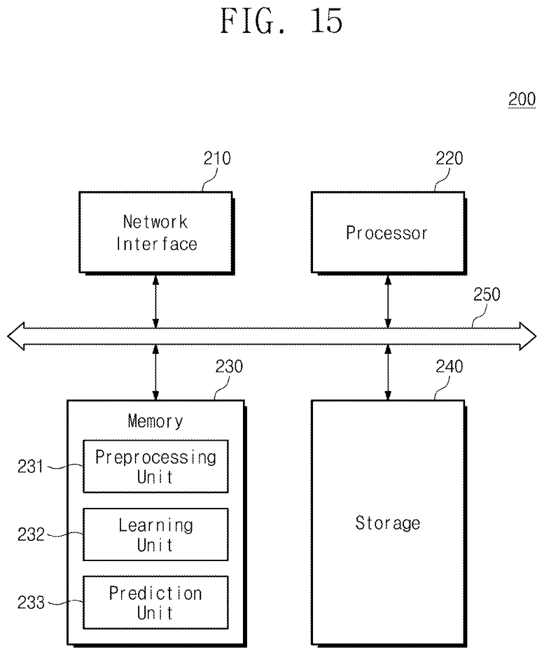

FIG. 15 is n block diagram of a time series data processing device of FIG. 1. The block diagram of FIG. 15 will be understood as a configuration for preprocessing time series data, generating a weight group, based on the preprocessed time series data, and generating a prediction result, based on the weight group. Referring to FIG. 15, a time series data processing device 200 may include a network interface 210, a processor 220, a memory 230, storage 240, and a bus 250. As an example, the time series data processing device 200 may be implemented as a server, but is not limited thereto.

The network interface 210 is configured to receive time series data provided from an external terminal (not illustrated) or a medical database through a network. The network interface 210 may provide the received time series data to the processor 220, the memory 230, or the storage 240 through the bus 250. In addition, the network interface 210 may be configured to provide a prediction result generated in response to the received time series data to an external terminal (not illustrated).

The processor 220 may function as a central processing unit of the time series data processing device 200. The processor 220 may perform a control operation and a calculation operation required to implement preprocessing and data analysis of the time series data processing device 200. For example, under the control of the processor 220, the network interface 210 may receive the time series data from an outside. Under the control of the processor 220, the calculation operation for generating a weight group of the feature distribution model may be performed, and a prediction result may be calculated using the feature distribution model. The processor 220 may operate by utilizing the computational space of the memory 230, and may read files for driving an operating system and executable files of an application from the storage 240. The processor 220 may execute the operating system and various applications.

The memory 230 may store data and process codes processed or scheduled to be processed by the processor 220. For example, the memory 230 may store time series data, information for performing a preprocessing operation of time series data, information for generating a weight group, information for calculating a prediction result, and information for constructing a feature distribution model. The memory 230 may be used as a main memory device of the time series data processing device 200. The memory 230 may include a Dynamic RAM (DRAM), a Static RAM (SRAM), a Phase-change RAM (PRAM), a Magnetic RAM (MRAM), a Ferroelectric RAM (FeRAM), a Resistive RAM (RRAM), etc.

A preprocessing unit 231, a learning unit 232, and a prediction unit 233 may be loaded into the memory 230 and may be executed. The preprocessing unit 231, the learning unit 232, and the prediction unit 233 correspond to the preprocessor 110, the learner 130, and the predictor 150 of FIG. 1, respectively. The preprocessing unit 231, the learning unit 232, and the prediction unit 233 may be a part of the computational space of the memory 230. In this case, the preprocessing unit 231, the learning unit 232, and the prediction unit 233 may be implemented as firmware or software. For example, the firmware may be stored in the storage 240 and loaded into the memory 230 when the firmware is executed. The processor 220 may execute the firmware loaded in the memory 230. The preprocessing unit 231 may be operated to preprocess the time series data under the control of the processor 220. The learning unit 232 may be operated to generate and train a feature distribution model by analyzing the preprocessed time series data under the control of the processor 220. The prediction unit 233 may be operated to generate a prediction result and a prediction basis, based on the feature distribution model under the control of the processor 220.

The storage 240 may store data generated for long-term storage by the operating system or applications, a file for driving the operating system, or an executable file of applications. For example, the storage 240 may store files for execution of the preprocessing unit 231, the learning unit 232, and the prediction unit 233. The storage 240 may be used as an auxiliary memory device of the time series data processing device 200. The storage 240 may include a flash memory, a phase-change RAM (PRAM), a magnetic RAM (MRAM), a ferroelectric RAM (FeRAM), and a resistive RAM (RRAM).

The bus 250 may provide a communication path between components of the time series data processing device 200. The network interface 210, the processor 220, the memory 230, and the storage 240 may exchange data with one another through the bus 250. The bus 250 may be configured to support various types of communication formats used in the time series data processing device 200.

According to an embodiment of the present disclosure, a time series data processing device and an operating method thereof may improve accuracy and reliability of a prediction result by improving irregular time intervals and uncertainty of a prediction time.

In addition, according to an embodiment of the present disclosure, a time series data processing device and an operating method thereof may provide an explainable prediction result by providing a basis and the validity for a prediction process of time series data using a feature distribution model.

The contents described above are specific embodiments for implementing the present disclosure. The present disclosure may include not only the embodiments described above but also embodiments in which a design is simply or easily capable of being changed. In addition, the present disclosure may also include technologies easily changed to be implemented using embodiments. Therefore, the scope of the present disclosure is not limited to the described embodiments but should be defined by the claims and their equivalents.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A time series data processing device comprising:
a preprocessor configured to generate interval data, based on a difference among each of a plurality of times on the basis of a last time of time series data, and to generate preprocessed data of the time series data; and
a learner configured to adjust a feature weight depending on a time and a feature of the time series data, based on the interval data and the preprocessed data, a time series weight depending on a correlation between the plurality of times and the last time, and a weight group of a feature distribution model for generating a prediction distribution of the time series data corresponding to the last time, and
wherein the weight group includes a first parameter for generating the feature weight, a second parameter for generating the time series weight, and a third parameter for generating the feature distribution model.

2. The time series data processing device of claim 1, wherein the preprocessor generates the preprocessed data by adding an interpolation value to a missing value of the time series data, and further generates masking data that distinguishes the missing value, and
wherein the learner adjusts the weight group, further based on the masking data.

3. The time series data processing device of claim 1, wherein the learner includes:
a feature learner configured to calculate the feature weight, based on the interval data, the preprocessed data, and the first parameter, and to generate a first learning result, based on the feature weight;
a time series learner configured to calculate the time series weight, based on the interval data, the first learning result, and the second parameter, and to generate a second learning result, based on the time series weight; and
a distribution learner configured to generate the prediction distribution, based on the second learning result and the third parameter, and
wherein the learner adjusts the weight group, based on the first learning result, the second learning result, and the prediction distribution.

4. The time series data processing device of claim 3, wherein the feature learner includes:
a missing value processor configured to generate first correction data of the preprocessed data, based on masking data that distinguishes a missing value of the preprocessed data;
a time processor configured to generate second correction data of the preprocessed data, based on the interval data;
a feature weight calculator configured to calculate the feature weight, based on the first parameter, the first correction data, and the second correction data; and
a feature weight applier configured to generate the first learning result by applying the feature weight to the preprocessed data.

5. The time series data processing device of claim 3, wherein the time series learner includes:
a time series weight calculator configured to calculate the time series weight, based on the interval data, the first learning result, and the second parameter; and
a time series weight applier configured to generate the second learning result by applying the time series weight to the preprocessed data.

6. The time series data processing device of claim 3, wherein the distribution learner includes:
a latent variable calculator configured to calculate a latent variable, based on the second learning result; and
a multiple distribution generator configured to generate the prediction distribution, based on the latent variable.

7. The time series data processing device of claim 1, wherein the learner encodes a result obtained by applying the feature weight to the preprocessed data, and calculates the time series weight, based on a correlation between the encoded result and the last time and a correlation between the encoded result and an encoded result of the last time.

8. The time series data processing device of claim 1, wherein the learner calculates a coefficient of the prediction distribution, an average of the prediction distribution, and a standard deviation of the prediction distribution, based on a learning result obtained by applying the time series weight to the preprocessed data.

9. The time series data processing device of claim 8, wherein the learner calculates a conditional probability of a prediction result for the preprocessed data on the basis of the prediction distribution, based on the coefficient, the average, and the standard deviation, and adjusts the weight group, based on the conditional probability.

10. A time series data processing device comprising:
a preprocessor configured to generate interval data, based on a difference among each of a plurality of times of time series data on the basis of a prediction time, and to generate preprocessed data of the time series data; and
a predictor configured to generate a feature weight depending on a time and a feature of the time series data, based on the interval data and the preprocessed data, to generate a time series weight depending on a correlation between the plurality of times and a last time, based on the feature weight and the interval data, and to calculate a prediction result corresponding to the prediction time and a reliability of the prediction result, based on the time series weight.

11. The time series data processing device of claim 10, wherein the preprocessor generates the preprocessed data by adding an interpolation value to a missing value of the time series data, and further generates masking data that distinguishes the missing value, and
wherein the predictor generates the feature weight, further based on the masking data.

12. The time series data processing device of claim 10, wherein the predictor includes:
a feature predictor configured to calculate the feature weight, based on the interval data, the preprocessed data, and a feature parameter, and to generate a first result, based on the feature weight;
a time series predictor configured to calculate the time series weight, based on the interval data, the first result, and a time series parameter, and to generate a second result, based on the time series weight; and
a distribution predictor configured to select at least some of prediction distributions, based on the second learning result and a distribution parameter, and to calculate the prediction result and the reliability, based on the selected prediction distributions.

13. The time series data processing device of claim 12, wherein the feature predictor includes:
a missing value processor configured to generate first correction data of the preprocessed data, based on masking data that distinguishes a missing value of the preprocessed data;
a time processor configured to generate second correction data of the preprocessed data, based on the interval data;
a feature weight calculator configured to generate calculate the feature weight, based on the feature parameter, the first correction data, and the second correction data; and
a feature weight applier configured to generate the first result by applying the feature weight to the preprocessed data.

14. The time series data processing device of claim 12, wherein the time series predictor includes:
a time series weight calculator configured to calculate the time series weight, based on the interval data, the first result, and the time series parameter; and
a time series weight applier configured to generate the second result by applying the time series weight to the preprocessed data.

15. The time series data processing device of claim 12, wherein the distribution predictor includes:
a latent variable calculator configured to calculate a latent variable, based on the second result;
a prediction value calculator configured to select at least some of the prediction distributions, based on the latent variable, and to calculate the prediction result, based on an average and a standard deviation of the selected prediction distributions; and
a reliability calculator configured to calculate the reliability, based on the standard deviation of the selected prediction distributions.

16. The time series data processing device of claim 10, wherein the predictor encodes a result obtained by applying the feature weight to the preprocessed data, and calculates the time series weight, based on a correlation between the encoded result and the prediction time and a correlation between the encoded result and an encoded result of the prediction time.

17. The time series data processing device of claim 10, wherein the predictor calculates coefficients, averages, and standard deviations of prediction distributions, based on a result obtained by applying the time series weight to the preprocessed data, selects at least some of the prediction distributions by sampling the coefficients, and generates the prediction result, based on the averages and the standard deviations of the selected prediction distributions.

18. A method of operating a time series data processing device, the method comprising:
generating preprocessed data obtained by preprocessing time series data;
generating interval data, based on a difference among each of a plurality of times of the time series data, on the basis of a prediction time;
generating a feature weight depending on a time and a feature of the time series data, based on the preprocessed data and the interval data;
generating a time series weight depending on a correlation between the plurality of times and the prediction time, based on a result of applying the feature weight and the interval data; and
generating characteristic information of prediction distributions, based on a result of applying the time series weight.

19. The method of claim 18, wherein the prediction time is a last time of the time series data, and
further comprising:
calculating a conditional probability of a prediction result for the preprocessed data, based on the characteristic information; and
adjusting a weight group of a feature distribution model for generating the prediction distributions, based on the conditional probability.

20. The method of claim 18, further comprising:
calculating a prediction result corresponding to the prediction time, based on the characteristic information; and
calculating a reliability of the prediction result, based on the characteristic information.
